(19) United States
(12) Patent Application Publication
(10) Pub. No.: US 2006/0135936 A1
(43) Pub. Date: Jun. 22, 2006

(54) Absorbent garment having sensory cue for line of weakness

(76) Inventors: Stacy N. Markovich, Appleton, WI (US); Suzanne M. Schmoker, Oshkosh, WI (US); Jeffery M. Tabor, Appleton, WI (US)

Correspondence Address: BRINKS HOFER GILSON & LIONE, P.O. BOX 10395, CHICAGO, IL, 60610, US

(21) Appl. No.: 11/020,482

(22) Filed: Dec. 22, 2004

(51) Int. Cl. A61F 13/15

(52) U.S. Cl. 604/386; 604/385.11

(57) ABSTRACT

An absorbent garment includes a body panel having a crotch edge and a waist edge, and first and second portions separated by a longitudinally extending line of weakness. The line of weakness extends between and terminates at the waist and crotch edges. The absorbent garment further includes a sensory cue directed at the line of weakness.

The present invention relates generally to an absorbent garment, and in particular, to an absorbent garment having a sensory cue identifying the location of a line of weakness formed in the garment.

### BACKGROUND

Absorbent garments can be configured in many different forms. For example, absorbent garments can be configured as a pant-type, pull-on garment, or as a diaper-type product that is drawn up between the legs and fastened about the waist with various fastening systems. Often, in the latter configuration, the fastening systems are configured to allow the user to detach and reattach various fasteners so as to provide a refastenable absorbent garment.

Some absorbent garments include a line of weakness, which is bridged by a fastening system. In this way, the absorbent garment can be used initially as a pull-on garment, but can be converted into a refastenable garment by breaking the line of weakness. Alternatively, the line of weakness can be broken before application to allow, for example, a caregiver to apply the garment to the user. With such garments, it can be difficult for the user or caregiver to locate the line of weakness, and/or to appreciate its capabilities. The problem of locating the line of weakness can be exacerbated for users or caregivers with impaired eyesight.

### SUMMARY

Briefly stated, in one aspect, the invention is directed to an absorbent garment including a body panel having first and second portions separated by a longitudinally extending line of weakness. The body panel further includes one or more sensory cues directed at the line(s) of weakness, wherein the sensory cue(s) provide indicia as to the location of the line(s) of weakness. A fastener member bridges the line of weakness. The sensory cue provides indicia to the user about the location and/or function of the line of weakness, thereby facilitating the use of the line of weakness.

In one embodiment, a second body panel includes opposite side edges joined to side edges of the first body panel to form a side seam.

In various embodiments, the sensory cue can include one or both of a visual cue or a tactile cue, including for example and without limitation colors, patterns, embossments, etc. In one embodiment, the sensory cue is more pronounced at a waist edge of the front body panel. In various embodiments, the sensory cue is located along the entire length of the line of weakness, or alternatively is located adjacent a waist edge and does not extend past a midpoint of the line of weakness between the waist edge and a crotch edge. The sensory cue can be positioned on one or more of the first or second portions of the body panel or on the fastener member.

In another aspect, an absorbent garment includes a body panel having a crotch edge and a waist edge, and first and second portions separated by a longitudinally extending line of weakness. The line of weakness extends between and terminates at the waist and crotch edges. The body panel further includes a sensory cue directed at the line of weakness, wherein the sensory cue is more pronounced adjacent the waist edge.

The present inventions will be best understood by reference to the following detailed description taken in conjunction with the accompanying drawings.

### BRIEF DESCRIPTION OF THE DRAWINGS

Many of the features and dimensions portrayed in the drawings, and in particular the presentation of layer thicknesses and the like, have been somewhat exaggerated for the sake of illustration and clarity.

FIG. 1 is a front view of one embodiment of an absorbent garment in a fastened configuration.

FIG. 2 is a bodyside plan view of one embodiment of an absorbent garment in an unfastened flat configuration.

FIG. 3 is a garment side plan view of stream of absorbent garments in an unfastened flat configuration.

FIG. 4A is a first embodiment of a sensory cue.

FIG. 4BA is a second embodiment of a sensory cue.

FIG. 4C is a third embodiment of a sensory cue.

FIG. 4D is a fourth embodiment of a sensory cue.

FIG. 4E is a fifth embodiment of a sensory cue.

FIG. 4F is a sixth embodiment of a sensory cue.

### DETAILED DESCRIPTION OF THE PRESENTLY PREFERRED EMBODIMENTS

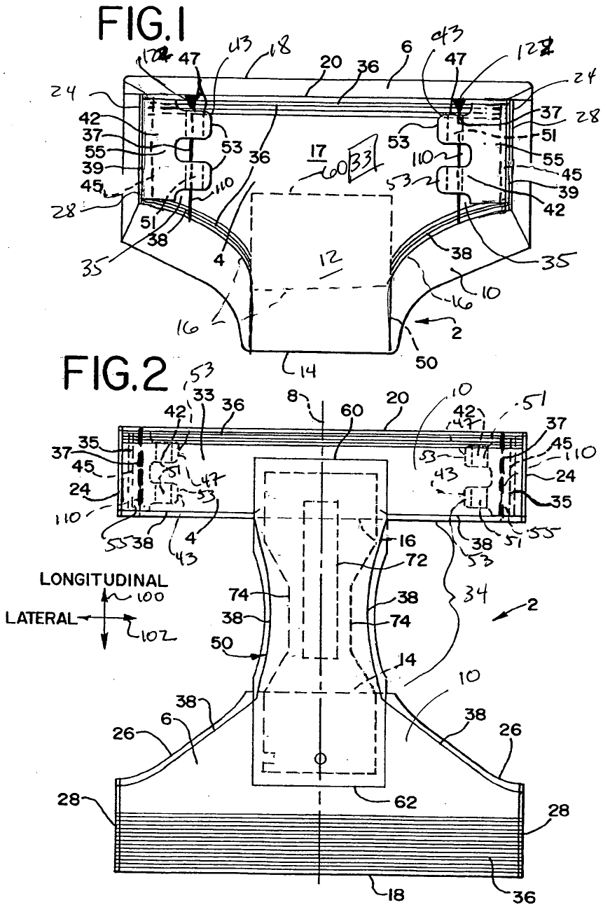

With reference to FIG. 2, it should be understood that the term “longitudinal,” as used herein, means of or relating to length or the lengthwise direction 100, and in particular, the direction running between the front and back of the user. The term “laterally,” as used herein means situated on, directed toward or running from side to side, and in particular, a direction 102 running from the left to the right of a user, and vice versa. The terms “upper,” “lower,” “inner,” and “outer” as used herein are intended to indicate the direction relative to the user wearing an absorbent garment over the crotch region, while the terms “inboard” and “outboard” refer to the directions relative to a centerline 8 of the garment. For example, the terms “inner” and “upper” refer to a “bodyside,” which means the side closest to the body of the user, while the terms “outer” and “lower” refer to a “garment side.”

The term “bodyside” should not be interpreted to mean in contact with the body of the user, but rather simply means the side that would face toward the body of the user, regardless of whether the absorbent garment is actually being worn by the user and regardless of whether there are or may be intervening layers between the component and the body of the user. Likewise, the term “garment side” should not be interpreted to mean in contact with the garments of the user, but rather simply means the side that faces away from the body of the user, and therefore toward any outer garments that may be worn by the user, regardless of whether the absorbent garment is actually being worn by a user, regardless of whether any such outer garments are actually worn and regardless of whether there may be intervening layers between the component and any outer garment.

Referring to FIGS. 1-3, an absorbent garment 2 includes a first, front body panel 4 and a second, rear body panel 6. As shown in FIG. 3, a stream 108 of absorbent garments 2 is shown prior to a front and rear body panel web 104, 106 being severed to create discrete absorbent garments. Referring to FIGS. 1-3, the first and second body panels each have an inner, bodyside surface 10 and an outer, garment side surface 12. The first, front body panel 4 has opposed first and second terminal edges 16 and 20, defining respectively a crotch and waist edge of the front body panel. Likewise, the second, rear body panel 6 has opposed first and second terminal edges 14 and 18, defining respectively a crotch and waist edge of the rear body panel. Each of the first and second body panels has an outboard edge 24, 28 formed along the outer periphery of laterally opposed side portions of the first and second body panel.

As shown in FIGS. 2 and 3, the second body panel 6 also includes opposite tapered edges 26. The first terminal edges 14, 16 of the first and second body panels are longitudinally spaced to form an opening 34 therebetween in the crotch region of the garment, while the second terminal edges 20, 18 of the first and second body panels form front and back waist edges respectively. The terminal crotch edge 16 of the front body panel can be linear between the outboard edges 24 as shown in FIGS. 2 and 3, or can include tapered portions as shown in FIG. 1. Likewise, the terminal crotch edge of the rear body panel can be linear between the outboard edges.

Referring to FIGS. 1-3, one or more, and desirably a plurality, meaning two or more, laterally extending elastic elements 36 are secured to each of the first and second body panels along the terminal edges 20, 18. In one exemplary embodiment, a plurality of laterally extending elastic elements is longitudinally spaced across a portion of the waist region of the rear body panel 6. It should be understood, that in an alternative embodiment, one or more separate waist bands, with or without elastic elements, can be secured to one or both of the rear and front body panels, preferably along the upper terminal edges thereof.

The front body panel 4 also has one or more elastic elements 36 extending laterally along the outer terminal edge 20. In one embodiment, a non-elasticized area is formed between the elastic elements 36 extending along the upper waist portion and elastic elements 38 extending along the lower terminal edges 14, 16 defining the leg opening. One or more leg elastic elements 38 can be secured along the inner terminal edges of the body panels 4, 6 and an absorbent composite 50 to form a gasket with the leg of the user. The various waist and leg elastic elements can be formed from rubber or other elastomeric materials. One suitable material is a Lycra® elastic material.

Each body panel is preferably formed as a composite or laminate material comprising at least two non-woven layers, otherwise referred to as substrates or laminates, with the plurality of elastic strands 38, 36 sandwiched therebetween. The two or more layers are bonded with various adhesives, such as hot melt, or by other techniques, including for example and without limitation ultrasonic bonding and heat pressure sealing. In this way, the body panels are preferably made of a relatively homogenous non-woven material, whether made of one or more layers or substrates, and preferably without any additional film materials or other types of materials being laminated thereto. As such, the body panels can be made with a relatively low basis weight, yet still exhibit requisite strength properties while remaining relatively soft to the touch. It should be understood that the body panels can be made of a single layer or substrate of non-woven material, or can be comprised of more than two layers or substrates. Of course, it should be understood that the body panels can be made up at least in part from other materials, including various knitted or woven fabrics, polymer films, laminates and the like. In one embodiment, the non-woven layers or substrates are made by spunbonding.

Referring to FIGS. 1-3, fastening members or tabs 42 are attached and extend laterally inboard from the outboard side edge 24 of the front body panel 4 from an attachment location 45. The front body panel 4 includes a middle portion 33, or landing member, and opposite side portions 35. Opposite longitudinally extending lines of weakness 37 separate the middle, landing member portion from the opposite side portions, such that the side portions are initially breakably attached to opposite sides of the landing member portion. The lines of weakness 37 can comprise a perforation or other series of cuts that allow a user or the manufacturer to separate the side portions from the middle portion. In other embodiments, the line of weakness can be formed by chemically weakening a portion of the body panel. The absorbent garment can be broken along the line of weakness 37 after the garment is applied to a user, or beforehand. Preferably, the fastening members 42 are secured to the garment-side surface of the side portions 35 between the side edge 24 of the front body panel and the line of weakness 37. The fastening members 42 bridge the lines of weakness 37. It should be understood that in one embodiment, only a single line of weakness is provided, while in other embodiments, more than two lines of weakness are provided. The lines of weakness can be positioned along any portion of the first and/or second body panels, allowing the user to separate one portion from another.

It should be understood that in other embodiments the fastening members can be secured to the rear body panel and engage the front body panel or, conversely, can be secured to the front body panel and engage the rear body panel. In other embodiments, the line of weakness is formed in the rear body panel. In one embodiment, the fastening members are fixedly secured to the outer, garment-side surface of the front and/or rear body panels, and releasably engage the outer, garment-side surface of the front and/or rear body panels, although it should be understood that the fastening members could be fixedly secured to an inner body-side surface of front and/or rear body panels and releasably engage an inner, body-side surface of the front and/or rear body panels.

As shown in FIGS. 1-3, the lines of weakness 37 extend between the terminal crotch and waist edges 14, 18, 16, 20 of the front body panel. In one embodiment, shown in FIG. 3, the lines of weakness 37 are formed as an arc, or curvilinear line, with a portion of the curve overlying or aligned with a curved portion of the fastening member. Of course, it should be understood that the lines of weakness can have many different shapes, including without limitation a straight line, a serpentine line, a zig-zag line, etc.

Referring to FIGS. 1-3, the front body panel 4 includes a sensory cue 110, 118, 122 directed at the line of weakness 37. The sensory cue provides indicia to the user about the location of the line of weakness. The term “directed at” does not require that the sensory cue necessarily contact, overlap or run all of the way to the line of weakness 37. In one embodiment, the sensory cue comprises a visual cue. The visual cue can take many forms, and can include one or more colors, shapes, graphics, text, alpha-numeric characters, and/or patterns, including indicia formed by dying, printing and/or embossing, or by otherwise altering the relative texture of the body panel. The cue can further be reflective and/or glow in the dark such that the user can access the line of weakness in poorly lit conditions.

For example, in the embodiments of FIGS. 1-3 and 4A-E, the visual cue 110, 112, 114, 118, 120 is configured as various decorative patterns applied along the entirety of the line of weakness 37 between the terminal crotch and waist edges 16, 20. In one suitable embodiment, the pattern is formed by repeating pictorials. Of course, it should be understood that the pattern can be any combination of repeating pictorials, lines, shapes, characters, etc. In one embodiment (not shown), the visual cue is configured as providing the side portion of the body panel with a first color or pattern and the middle/landing portion of the body panel with a second color or pattern that is different than the first color, meaning the colors have a different hue. Alternatively, a portion of the both the middle and side portions can be configured with a different color along the line of weakness. For example, where the line of weakness is formed by a chemical weakening, the line of weakness can be identified by a dye, which can be added to the chemical weakener. One or more colors may also be different by virtue of having a different luminosity and/or saturation/vividness. Saturation/vividness is the intensity of the color from pale to dark. The portions may also have a different gloss/finish, from a matte finish, which tends to diffuse or scatter light, to a gloss finish, which is specular.

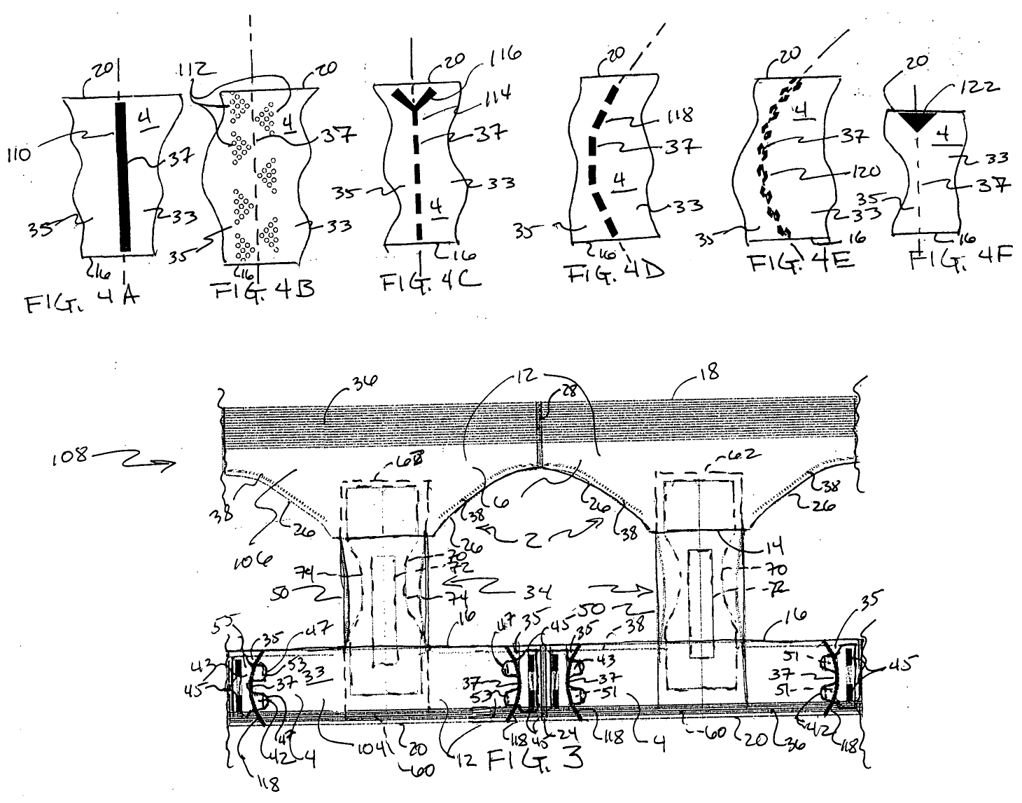

As shown in FIG. 4B, the sensory cue 112 can be formed as a pattern along opposite sides of the line of weakness 37 on both the side portion 35 and middle portion 33 of the body panel 14. Alternatively, the pattern (not shown) can be formed along only one side of the line of weakness on one of the side portion or middle portion. As shown in FIGS. 1, 4C and 4F, the sensory cue 122, 116 is more pronounced adjacent the waist edge 20 of the body panel 4. The term “pronounced,” as used herein, means more strongly or distinctively marked, whether in shape, boldness, tone, size, roughness, etc. than a remainder or other portion of the cue. For example, the pattern in FIGS. 1 and 4C is more pronounced at the waist edge by way of a first portion 122, 116 having a different shape and relative size, e.g., width, as compared with the remainder of the sensory cue 110, 114. As shown in FIG. 4F, the cue 122 is more pronounced at the waist edge by virtue of its absence elsewhere. As shown in FIG. 1, the sensory cue is configured as a combination of sensory cues 110 and 122 shown in FIGS. 4A and 4F.

In the embodiments of FIGS. 1, 4C and 4F, at least a portion of the sensory cue 116, 122 is configured as a pattern directed away from the waist edge 20 toward the crotch edge. The pattern of FIG. 4C includes a downwardly directed arrow 116, which is positioned adjacent the waist edge 120, and a dashed line 114 extending from the point of the arrow 116 down at least a portion of the line of weakness 37, and in one embodiment along the entire extent of the line of weakness. The overall pattern of FIG. 4C resembles a zipper, which provides instructional indicia to the user about what to do at the line of weakness. For example, and without limitation, the zipper indicia 114, 116 informs the user that the line of weakness 37 can be undone, most desirably from the top, or waist edge 20, down to the bottom, or crotch edge 16.

Other sensory cues are configured as solid lines 110 (FIG. 4A), dashed lines 118 (FIG. 4D), staggered laterally directed arrows 112 (FIG. 4B), staggered footprints 120 (FIG. 4E), or a simple arrow 122 (FIG. 4F) located adjacent one of the crotch and/or waist edges 20, 16. In the embodiment of FIG. 4F, the arrow 122 is directed away from the waist edge 20, toward the crotch edge, thereby informing the user of how to undo the line of weakness 37. In any of the embodiments, and in particular the embodiment of FIG. 4F, the sensory cue 122 is located adjacent only one edge 20 and/or does not extend beyond a midpoint between the waist and crotch edges 20, 16. For example, in various embodiments, the length of the cue ranges from about ¼ inch to the full length of the line of weakness, and the width of the cue ranges from about ⅛ inch to about two (2) inches. It should be understood that a pair of arrows (not shown), or other indicia, could be located adjacent both the waist and crotch edges, and further that any of the other embodiments could extend along the entirety, or only a portion, of the line of weakness.

In one embodiment, the sensory cue is configured at least in part as a tactile cue, which can include for example and without limitation embossing, or otherwise altering the relative texture of the body panel. Of course, it should be understood that the sensory cue can take many forms, and can include various combinations of colors, patterns, shading etc., including visual and tactile embodiments. In some embodiments, the sensory cue includes one or both of a visual cue and a tactile cue. For example, one or both of the side and middle portions, or portions of both along the line of weakness, can have different textures so as to form a tactile cue. In some embodiments, the tactile cue is formed by an embossment, or different embossments. Similarly, a separate piece of material applied to one or both of the side or middle portions may have a different texture or feel than the other thereof, thereby providing a tactile cue as to the location of the line of weakness. In one embodiment, the surface texture of the outercover material is changed so that the user can identify the location of the line of weakness by feel. This change can be achieved for example and without limitation by melting the region of the material adjacent the line of weakness, or by hardening the material, for example by adding adhesives etc.

In one embodiment, the sensory cue, such as a print or dye, is applied to an interior surface of one of the layers of the body panel, with an outer layer thereof being substantially transparent, defined as allowing a user to see the underlying cue. In this way, the ink, or other dye or print medium, is not in direct contact with either the body of the user or any outer garment.

Referring to FIG. 1, the opposite side edges 24 of the front body panel 4 are joined to the opposite side edges 28 of the rear body panel 6 to form a seam 39. In this way, prior to the breaking of the line of weakness 37, the absorbent garment can be configured as a pant-like garment, which can be pulled over the legs of the user. After the garment is applied to the user, the lines of weakness 37 can be broken, if desired, or left intact, as the fasteners 42 are adjusted to fit the garment to the user. If desired, the lines of weakness can be broken prior to securing the garment to the user, for example when the user is bed-ridden. In this configuration, the garment is laid beneath the user and is secured to the user with the fastening tabs 47. By providing the side portions 35, and by connecting the fastening tabs 47 to the front panel 4, instead of the rear body panel 6, the tabs are located at the front of the user so as to not provide discomfort to the user when lying on their backs.

In one embodiment, a portion or all of the fastening members 42 or tabs 47 are provided with sensory cue indicia similar to the cue indicia located on the body panel along the line of weakness such that the fastener members 42 and line of weakness 37 are understood to be an overall system that provides maximum adjustability. In one embodiment, the sensory cue is applied only to the fastener member and is directed toward the line of weakness lying thereunder, for example by directing the sensory cue at the line of weakness at the edges of the fastener member, or by forming a cue along the fastener member that corresponds to and is aligned with the underlying line of weakness.

It should be understood that the front and rear body panels can be made as a unitary member that extends along the crotch from the front to back and with the sides thereof connected to form side seams. Alternatively, the front and rear body panels can be formed integrally, for example as one panel extending around the waist and hips of the user, and then secured, for example by a single side seam. In one embodiment, the side seam defines the line of weakness.

Preferably, as shown in FIGS. 1-3, the fastening members 42 comprise a carrier member 43 that is formed in a generally side-ways, “U” shape, with a vertical extending base member 55 and a pair of laterally extending and longitudinally spaced tab members 47. It should be understood that the carrier member could include one or more than two tab members. The carrier members are preferably fixedly secured to the side portions of the body panel 4 with adhesive bonds 45, sonic bonds, thermal bonds, pinning, stitching or other known types of attachment, as shown for example in FIGS. 2 and 3. In alternative embodiments, the fastening members can be fixedly secured to the rear panel, or to one or both of the front and rear panels, e.g., at the seam 39.

Each of the tab members 47 comprises an engagement portion, which includes in various embodiments an array of hooks, various adhesives, such as pressure sensitive adhesives, buttons, zippers and other releasable and reattachable fastening devices.

In one embodiment, each fastening member 42 is comprised of two separate, longitudinally spaced tab members 47. In any of the embodiments, the two or more tab members provides a pant like fit that controls the waist and leg openings in the front and back of the garment, and also allows the user to adjust the fit of the garment without totally undoing the garment. For example, the user can release one of the tab members and refasten it without undoing the other tab member on that side.

In one embodiment, a hook-type fastener member 51, or hook strip, is secured to the carrier member 43 with adhesive, ultrasonic bonding, stitching or other known attachment devices. The end portion 53 or tip of the carrier member can be left uncovered by the fastener member 51, such that it can be lifted or flexed and grasped by a user as they disengage or peel back the fastener member. It should be understood that the term “hook” as used herein means any element capable of engaging another element, and is not intended to limit the form of the engaging elements, for example to include only “hooks,” but rather encompasses any form shape of engaging element.

When the absorbent garment is secured to the user, the fastening tabs 42 secured to the side portions of the front body panels 4 releasably engage or are otherwise connected to the middle portion of the body panels 4. The landing portion 33 of the front body panel 4 can be configured with an additional material, or landing patch, secured to the garment side thereof and which includes a loop material in one exemplary embodiment.

Referring again to FIGS. 1-3, the absorbent garment includes an absorbent composite 50 having first and second longitudinally opposed terminal end edges 60, 62. The absorbent composite includes a substantially liquid permeable topsheet, or liner, and a substantially liquid impermeable backsheet, or outer cover. A retention portion 70 is disposed or sandwiched between the topsheet and the backsheet, which are connected. The topsheet, backsheet and other components of the absorbent composite 50 can be joined for example with adhesive bonds, sonic bonds, thermal bonds, pinning, stitching or any other attachment techniques known in the art, as well as combinations thereof. For example, a uniform continuous layer of adhesive, a patterned layer of adhesive, a sprayed pattern of adhesive or any array of lines, swirls or spots of construction bonds may be used to join the topsheet and backsheet, or any of the other components described herein.

Additional layers, including for example, a surge layer 72, can also be incorporated into the absorbent composite. Preferably, the surge layer does not run the entire length of the absorbent composite and is shorter than the retention portion. The topsheet can be indirectly joined to the backsheet by affixing the topsheet to intermediate layers, such as the surge layer or retention portion, which in turn is affixed to the backsheet.

The backsheet is preferably liquid impermeable, but may be liquid permeable, e.g., when an additional barrier layer is used with the retention portion. The backsheet typically provides the outercover of the article. Optionally, however, the article may include a separate outercover component member, which is additional to the backsheet. The outercover can be joined, for example, to one or more of the absorbent composite and/or body panels.

The retention portion 70 is preferably made of an absorbent material, which tends to swell or expand as it absorbs liquid excreted or exuded by the user. For example, the absorbent material can be made of airformed, airlaid and/or wetlaid composites of fibers and high absorbency materials, referred to as superabsorbents. In addition, various foams, absorbent films, and superabsorbent fabrics can be used as an absorbent material.

The retention portion 70 has laterally opposed side edges 74 and preferably is made of a single layer of material. The retention portion preferably has an hour-glass shape with enlarged end regions. Alternatively, the retention portion can include a folded or multi-layered configuration. The retention portion preferably has a length substantially equal to, or slightly shorter than, the length of the absorbent composite. The retention portion can include one or more barrier layers attached to the absorbent material. In one embodiment, an upper tissue substrate is disposed adjacent the retention portion. Alternatively, a lower tissue substrate can be disposed adjacent an opposite side of the retention portion, or the tissue can completely envelope the retention position.

Referring to FIGS. 1-3, the opposite garment side of the end regions of the absorbent composite, and in particular, the outer, garment side surface of the backsheet, are secured to the bodyside surface of the longitudinally opposed crotch ends of the first and second body panels 4, 6. Conversely, the absorbent composite can be secured to the garment side surface of the body panels. It should be understood that the absorbent composite can be secured using any of the methods of attachment described above, including for example various adhesives, stitching or other bonding methods. The absorbent composite can be secured to the body panels with any configuration of attachment lines, swirls, patterns, spots, etc., or can be a full and continuous attachment therebetween.

One suitable absorbent garment construction having front and rear body panels with lines of weakness is the Depend® refastenable underwear available from Kimberly-Clark Corporation, located in Neenah, Wisconsin and the Assignee of the present application. In addition, various absorbent garment constructions are disclosed for example and without limitation in U.S. Patent Application Publication US 2003/0135191A1 (Ser. No. 09/899,808), filed Jul. 5, 2001, and U.S. Patent Application Publication US 2003/0088223 (Ser. No. 09/834,682), filed Apr. 13, 2001, the entire disclosures of which are hereby incorporated herein by reference.

Although the present invention has been described with reference to preferred embodiments, those skilled in the art will recognize that changes may be made in form and detail without departing from the spirit and scope of the invention. As such, it is intended that the foregoing detailed description be regarded as illustrative rather than limiting and that it is the appended claims, including all equivalents thereof, which are intended to define the scope of the invention.

What is claimed is:

1. An absorbent garment comprising:
a body panel having a crotch edge and a waist edge and comprising first and second portions separated by at least one longitudinally extending line of weakness, wherein said line of weakness extends between said waist and crotch edges;
a fastener member bridging said line of weakness; and
at least one sensory cue directed at said line of weakness, wherein said at least one sensory cue provides indicia as to the location of said line of weakness.

2. The absorbent garment of claim 1 wherein said body panel comprises a first body panel comprising opposite side edges and said first and second portions, and further comprising a second body panel comprising opposite side edges, wherein said opposite side edges of said first and second body panels are joined to form side seams.

3. The absorbent garment of claim 2 wherein said first portion defines a first side portion and said second portion defines a landing portion, and further comprising a second side portion, wherein said first and second side portions define said opposite side edges and said landing portion extends between said first and second side portions, and wherein said line of weakness comprises a first line of weakness separating said first side portion and said landing portion, and further comprising a second line of weakness separating said second side portion and said landing portion, and wherein said first and second lines of weakness extend longitudinally across an entire length of said first body panel between said first and second side portions and said landing portion.

4. The absorbent garment of claim 1 wherein said line of weakness comprises a perforation.

5. The absorbent garment of claim 1 wherein said sensory cue comprises a visual cue disposed on at least one of said first portion, said second portion and said fastener member.

6. The absorbent garment of claim 5 wherein said visual cue comprises a first color different from a second color of at least one of said first and second portions.

7. The absorbent garment of claim 5 wherein said visual cue comprises a pattern printed on at least one of said first and second portions.

8. The absorbent garment of claim 1 wherein said sensory cue is more pronounced at a waist edge of said body panel.

9. The absorbent garment of claim 8 wherein said sensory cue comprises a pattern directed away from said waist edge.

10. The absorbent garment of claim 8 wherein said sensory cue comprises an arrow directed away from said waist edge.

11. The absorbent garment of claim 1 wherein said sensory cue comprises a tactile cue.

12. The absorbent garment of claim 11 wherein said tactile cue comprises an embossment.

13. The absorbent garment of claim 1 wherein said sensory cue is located along the entire length of said line of weakness.

14. The absorbent garment of claim 1 wherein said sensory cue is located only adjacent a waist edge of said body panel.

15. The absorbent garment of claim 1 wherein at least a portion of said line of weakness is curvilinear.

16. The absorbent garment of claim 1 wherein said body panel comprises at least inner and outer layers, wherein said sensory cue comprises a visual cue applied to an interior surface of at least one of said inner and outer layers between said inner and outer layers, wherein said outer layer is sufficiently transparent such that said sensory cue is visible to the user.

17. An absorbent garment comprising:
a body panel having a crotch edge and a waist edge and comprising first and second portions separated by a longitudinally extending line of weakness, wherein said line of weakness extends between and terminates at said waist and crotch edges, and said body panel further comprising a sensory cue directed at said line of weakness, wherein said sensory cue is more pronounced adjacent said waist edge.

18. The absorbent garment of claim 17 further comprising a fastener member bridging said line of weakness.

19. The absorbent garment of claim 17 wherein said sensory cue is located adjacent said waist edge and does not extend past a midpoint of said line of weakness between said waist and crotch edges.

20. The absorbent garment of claim 17 wherein said sensory cue comprises an arrow directed away from said waist edge.
